(19) United States
(12) Patent Application Publication
(10) Pub. No.: US 2022/0307971 A1
(43) Pub. Date: Sep. 29, 2022

(54) SYSTEMS AND METHODS FOR PHENOTYPING

(71) Applicants: Carmel Haifa University Economic Corporation Ltd., Haifa (IL); Evogene Ltd., Rehovot (IL); Elbit Systems Land and C4I Ltd., Netanya (IL); Hazera Seeds Ltd., Shikmim (IL); Opgal Optronics Industries Ltd., Karmiel (IL); Rahan Meristem (1998) Ltd., Western Galilee (IL); Sensilize Ltd., Tel Aviv (IL); The State of Israel, Ministry of Agriculture & Rural Development, Agricultural Research Organization, Rishon Lezion (IL); Technion Research and Development Foundation Limited, Haifa (IL)

(72) Inventors: Lior Coen, Petach-Tikva (IL); Victor Alchanatis, Mazkeret Batya (IL); Ohsry Markovich, Kfar Vradim (IL); Yoav Zur, Kohav Michael Sobel (IL); Daniel Koster, Rehovot (IL); Yogev Montekyo, Rishon Lezion (IL); Hagai Karchi, Sitria (IL); Ilya Leizerson, Haifa (IL); Sharone Aloni, Beit Herut (IL); Anna Brook, Haifa (IL); Zur Granevitze, Mazkeret Batya (IL); Yaron Honen, Haifa (IL); Alon Zvirin, Haifa (IL); Ron Kimmel, Haifa (IL)

(21) Appl. No.: 17/610,863

(22) PCT Filed: May 13, 2020
(86) PCT No.: PCT/IL2020/050515

§ 371 Date: Nov. 12, 2021

Related U.S. Application Data
(60) Provisional application No. 62/846,764, filed on May 13, 2019.

(51) Int. Cl. G01N 21/27; G01N 33/02; G01S 17/894; G01S 17/86; G01N 21/25; G06V 10/143; G06V 10/147; G06V 10/80; G06V 10/40; G06V 10/20; G06T 7/30; H04N 5/247; H04N 5/232; G06V 10/774; G06T 7/00; G06V 20/10

(52) U.S. Cl. CPC G01N 21/27; G01N 33/025; G01N 21/274; G01S 17/894; G01S 17/86; G01N 21/251; G06V 10/143; G06V 10/147; G06V 10/803; G06V 10/40; G06V 10/20; G06T 7/30; H04N 5/247; H04N 5/23203; G06V 10/774; G06T 7/0012; G06V 20/188; G01N 2021/8466

(57) ABSTRACT

The present invention relates to the field of phenotyping, particularly to systems and methods for collecting, retrieval and processing of data for accurate and sensitive analysis and prediction of a phenotype of an object, particularly of a plant.

### FIELD OF THE INVENTION

The present invention relates to the field of phenotyping, particularly to systems and methods for collecting, retrieval and processing of data for accurate and sensitive analysis and prediction of a phenotype of an object, particularly a plant.

### BACKGROUND OF THE INVENTION

The constant increase in the world population and the demand for high quality food without negatively affecting the environment, creates the needs to develop technological means for use in agriculture and Eco culture. Tools for precision farm management with the goal of optimizing returns on investment while preserving resources are required.

In some situations, agricultural management may relate to plant breeding, developing new plant types, planning location and density of future plantations, planning for selling or otherwise using the expected crops, or the like. These activities may be performed by agronomists consulting to the land owner or user, the agronomists executing visual and other inspections of the plants and the environment and providing recommendations. However, as in other fields, the reliance on manual labor significantly limits the capacity and response time which may lead to sub-optimal treatment and lower profits.

The process of crop phenotyping, including the extraction of visual traits from plants, allows crop examination and inferring important properties concerning the crop status (Araus, J. L ET AL. 2014. Trends in plant science 19, 52-61). Crops phenotyping relies on non-destructive collection of data from plants over time. Developing precision management requires tools for collecting plant phenotypic data, environmental data and computational environment enabling high throughput processing of the data received. Translation of the processed data to an agriculture and/or eco-culture recommendation is further required.

There is an ongoing effort to develop systems and methods for precision agriculture based on plant imaging. For example, U.S. Patent Application Publication No. 2004/0264761 discloses a system and method for creating 3-dimensional agricultural field scene maps comprising producing a pair of images using a stereo camera and creating a disparity images based on the pair of images, the disparity image being a 3-dimensional representation of the stereo images. Coordinate arrays can be produced from the disparity image and the coordinate arrays can be used to render a 3-dimensional local map of the agricultural field scene. Global maps can also be made by using geographic location information associated with various local maps to fuse together multiple local maps into a 3-dimensional global representation of the field scene.

U.S. Patent Application Publication No. 2013/0325346 discloses systems and methods for monitoring agricultural products, particularly fruit production, plant growth, and plant vitality. In some embodiments, the invention provides systems and methods for a) determining the diameter and/or circumference of a tree trunk or vine stem, determining the overall height of each tree or vine, determining the overall volume of each tree or vine, determining the leaf density and average leaf color of each tree or vine; b) determining the geographical location of each plant and attaches a unique identifier to each plant or vine; c) determining the predicted yield from identified blossom and fruit; and d) providing yield and harvest date predictions or other information to end users using a user interface.

International (PCT) Patent Application Publication No. WO 2016/181403 discloses an automated dynamic adaptive differential agricultural cultivation system, constituted of: a sensor input module arranged to receive signals from each of a plurality of first sensors positioned in a plurality of zones of a first field; a multiple field input module arranged to receive information associated with second sensors from a plurality of fields; a dynamic adaptation module arranged, for each of the first sensors of the first field, to compare information derived from the signals received from the respective first sensor with a portion of the information received by the multiple field input module and output information associated with the outcome of the comparison; a differential cultivation determination module arranged, responsive to the output information of the dynamic adaptation module, to determine a unique cultivation plan for each zone of the first field; and an output module arranged to output a first function of the determined unique cultivation plans.

International (PCT) Patent Application Publication No. WO/2016/123201 discloses systems, devices, and methods for data-driven precision agriculture through close-range remote sensing with a versatile imaging system. This imaging system can be deployed onboard low-flying unmanned aerial vehicles (UAVs) and/or carried by human scouts. The technology stack can include methods for extracting actionable intelligence from the rich datasets acquired by the imaging system, as well as visualization techniques for efficient analysis of the derived data products.

U.S. Patent Applications Publication No. 2016/0148104 and 2017/0161560 disclose system and method for automatic plant monitoring, comprising identifying at least one test input respective of a test area, wherein the test area includes at least one part of a plant; and generating a plant condition prediction based on the at least one test input and on a prediction model, wherein the prediction model is based on a training set including at least one training input and at least one training output, wherein each training output corresponds to a training input. The plant condition to be predicted include a current disease, insect and pest activity, deficiencies in elements, a future disease, a harvest yield, and a harvest time.

U.S. Pat. No. 10,182,214 discloses an agricultural monitoring system composed of an airborne imaging sensor, configured and operable to acquire image data at sub-millimetric image resolution of parts of an agricultural area in which crops grow, and a communication module configured and operable to transmit to an external system image data content which is based on the image data acquired by the airborne imaging sensor. The system further comprises a connector operable to connect the imaging sensor and the communication module to an airborne platform.

Object segmentation, detection and classification based on image processing and data analyses is widely used in various fields of interest under laboratory conditions. However, there remain a need for systems and methods which can provide reproducible, high quality images under greenhouse or open filed conditions and for use thereof in decision support systems for precision agriculture.

### SUMMARY OF THE INVENTION

The present invention discloses systems and methods for determining and predicting phenotype(s) of a plant or of a plurality of plants. The phenotypes are useful for managing the plant growth, particularly for precise management of agricultural practices, for example, breeding, fertilization, stress management including disease control, and management of harvest and yield. The systems and methods of the invention may be based on, but not limited to data obtained during the growing season (presently obtained or recently obtained data) and on an engine having reference data and phenotypes, including engine trained to determine and/or predict a phenotype based on reference data previously obtained by the systems of the present invention and phenotypes corresponding to the reference data. The systems and methods of the present invention provide for phenotypes at meaningful agricultural time points, including, for example, very early detection of biotic as well as abiotic stresses, including detecting of stress symptoms before the symptoms of the stress are visible to the human eye or to a single Red- Green-Blue (RGB) camera. Advantageously, the system and methods of the present invention can capture the plant as a whole as well as plant parts and objects present on the plan parts, including, for example, the presence of insects or even insect eggs which predict a potential to develop a disease phenotype.

The present invention is based in part on a combination of (i) data obtained from a plurality of imaging sensors set at a predetermined geometrical relationship; (ii) means to effectively reduce variations in data readings resulting from the outdoor environmental conditions, sensors effects, and other factors including object positioning and angle of data acquisition; and (iii) computational methods of processing the data. The processed data synchronized and aligned across the various sensors are highly reproducible, enabling both—training an engine to set a phenotype, and using the trained engine or another engine to determine and/or predict a phenotype based on newly obtained processed data.

The invention may also utilize improvement of internal sensor data resolution and blurring correction.

According to one aspect, the present invention provides a system for detecting or predicting a phenotype of a plant, comprising:

a plurality of imaging sensors of different modalities selected from the group consisting of: a Red-Green-Blue (RGB) sensor; a multispectral sensor; a hyperspectral sensor; a depth sensor; a time-of-flight camera; a LIDAR; and a thermal sensor, the plurality of sensors mounted on a bracket at predetermined geometrical relationships;

a computing platform comprising at least one computer-readable storage medium and at least one processor for:

receiving data captured by the plurality of sensors, the data comprising at least two images of at least one part of a plant, the at least two images captured at a distance of between 0.05 m and 10 m from the plant;

preprocessing the at least two images in accordance with the predetermined geometrical relationship, to obtain unified data;

extracting features from the unified data; and

providing the features to an engine to obtain a phenotype of the plant.

According to certain embodiments, the engine is a trained neural network or a trained deep neural network.

According to certain embodiments, the processor is further adapted to display to a user an indicator helpful in verifying the reliability of the engine.

According to certain embodiments, the indicator helpful in verifying the reliability of the engine is a class activation map of the engine.

According to certain embodiments, the at least two images are captured at a distance of between 0.05 m and 5 m from the plant.

According to certain embodiments, the processor is further adapted to:

receive from at least one additional sensor additional data related to positioning and/or environmental conditions of the plant; and

process the at least two images using the additional data to eliminate effects generated by the environmental conditions and/or positioning to obtain at least two enhanced images before preprocessing.

According to certain embodiments, the preprocessing comprises preprocessing the at least two enhanced images.

According to certain embodiments, the at least one additional sensor is selected from the group consisting of: a light sensor, a global positioning system (GPS); a digital compass; a radiation sensor; a temperature sensor; a humidity sensor; a motion sensor; an air pressure sensor; a soil sensor; an inertial sensor and any combination thereof.

According to certain exemplary embodiments, the at least one additional sensor is a light sensor.

According to certain embodiments, preprocessing comprises at least one of: registration; segmentation; stitching; lighting correction; measurement correction; and resolution improvement.

According to certain embodiments, the preprocessing comprises registering the at least two enhanced images in accordance with the predetermined geometrical relationships.

According to certain embodiments, registering the at least two enhanced images comprises alignment of the at least two enhanced images.

Advantageously, the preprocessing according to the teachings of present invention provides for unified data enabling extracting the features in a highly accurate, reproducible manner. The percentage of prediction accuracy depends on the feature to be detected. According to certain embodiments, the accuracy of the feature prediction is at least 60%.

According to certain embodiments, the measurement correction comprises correction of data captured by at least one imaging sensor.

According to certain embodiments, the computing platform may also be operative in receiving information related to mutual orientation among the sensors. According to certain embodiments, the computing platform may also be operative in receiving information related to mutual orientation between the sensors and at least one of an illumination source and a plant.

According to certain embodiments, the plurality of imaging sensors comprises at least two of said imaging sensors. According to certain embodiments, the plurality of imaging sensors comprises two of said imaging sensors. According to certain embodiments, the plurality of imaging sensors consists of two of said imaging sensors.

According to certain embodiments, the plurality of imaging sensors comprises at least three of said imaging sensors.

According to certain embodiments, the plurality of imaging sensors comprises three of said imaging sensors. According to some embodiments, the plurality of imaging sensors consists of three of said imaging sensors.

The specific combination of the imaging sensors and optionally of the at least one additional sensor may be determined according to the task to be performed, including, for example, detecting or predicting a phenotype, the nature of the phenotype, the type and species of the plant or plurality of plants and the like.

According to certain embodiments, the plurality of imaging sensors comprises an RGB sensor and a multispectral sensor or a hyperspectral sensor. According to some embodiments, the plurality of imaging sensors consists of an RGB sensor and a multispectral sensor or a hyperspectral sensor. According to certain alternative embodiments, the plurality of imaging sensors comprises an RGB sensor and a thermal sensor. According to some embodiments, the plurality of imaging sensors consists of an RGB sensor and a thermal sensor. According to some embodiments, the plurality of imaging sensors comprises RGB sensor, thermal sensor and depth sensor. According to some embodiments, the plurality of imaging sensors consists of RGB sensor, thermal sensor and depth sensor.

According to certain exemplary embodiments, a combination of imaging sensors comprising an RGB sensor and multi spectral sensor or a combination of imaging sensors comprising an RGB sensor, a thermal sensor and a depth sensor provides for early detection of a phenotype of stress resulting from fertilizer deficiency, before stress symptoms are visible to the human eye or by a single RGB sensor. According to certain embodiments, an external lighting monitoring is added to the combination of imaging sensors.

According to certain embodiments, the at least two images can provide for distinguishing between plant parts and/or objects present on the plant part. According to certain embodiments, the objects are plant pests or parts thereof.

According to some embodiments, the RGB sensor can provide for distinguishing between plant parts and/or objects present on the plant part.

According to certain embodiments, multi-spectral and lighting sensors can provide for identifying significant signature differences between healthy and stress plants.

According to certain embodiments, RGB sensor may provide for detecting changes in leaf color, a depth sensor may provide for detecting changes in plant size and growth rate; and a thermal sensor may provide for detecting changes in transpiration. According to certain exemplary embodiments, combinations of the above can provide for early detection and predicting stress resulting from lack of water or lack of fertilizer.

According to certain embodiments, the plurality of imaging sensor provides at least one image of a plant part selected from the group consisting of a leaf, a petal, a flower, an inflorescent, a fruit, and parts thereof.

According to certain embodiments, each of the plurality of imaging sensors or of the at least one additional sensors is calibrated independently of other sensors. According to additional or alternative embodiments, the plurality of imaging sensors and the at least one additional sensor are calibrated as a whole. According to certain exemplary embodiments, at least one calibration is radiometric calibration.

According to certain embodiments, the preprocessing comprises at least one of: registration; segmentation; stitching; lighting correction; measurement correction; and resolution improvement.

According to certain embodiments, wherein the preprocessing comprises registering the at least two enhanced images in accordance with the predetermined geometrical relationships.

According to certain embodiments, registering the at least two enhanced images comprises alignment of the at least two enhanced images.

According to certain embodiments, the at least one additional sensor is selected from the group consisting of: a digital compass; a global positioning system (GPS); a light sensor for determining lightning conditions, such as a light intensity sensor; a radiation sensor; a temperature sensor; a humidity sensor; a motion sensor; an air pressure sensor; a soil sensor, and an inertial sensor. According to certain exemplary embodiments, the at least one additional sensor is a light sensor.

The at least one additional sensor can be located within the system or remote of the system. According to certain embodiments, the at least one additional sensor is located within the system, separate of the bracket mounted with the plurality of imaging sensors.

According to certain embodiments, the at least one additional sensor is located within the system on said bracket at predetermined geometrical relationships with the plurality of imaging sensors.

According to certain embodiments, the computing platform is located separate of the bracket mounted with the plurality of imaging sensors. According to certain embodiments, the computing platform is located on said bracket.

According to certain embodiments, the system further comprises a command and control unit for coordinating activation of the plurality of sensors; and operating the at least one processor in accordance with the plurality of sensors and with the at least one additional sensor. According to these embodiments, the command and control unit is further operative to perform at least one action selected from the group consisting of: setting a parameter of a sensor from the plurality of sensors; operating the at least one processor in accordance with a selected application; providing an indication to an activity status of a sensor from the plurality of sensors; providing an indication to a calibration status of a sensor from the plurality of sensors; and recommending to a user to calibrate a sensor from the plurality of sensors.

According to certain embodiments, the system further comprises a communication unit for communicating data from said plurality of sensors to the computing environment. The communication unit can be within the system or remote of the system.

According to certain embodiments, the system further comprises a cover and at least one light intensity sensor positioned on the cover for enabling, for example, radiometric calibration of the system.

The system of the present invention can be stationary, mounted on a manually held platform, or installed on a moving vehicle.

The complex interaction between a plant genotype and its environment controls the biophysical properties of the plant, manifested in observable traits, i.e., the plant phenotype or phenome. The system of the present invention can be used to determine and/or predict a plant phenotype of agricultural or ecological importance as long as the phenotype is associated with imagery data that may be obtained from the plant. Advantageously, the system of the invention enables detection of a phenotype at an early stage, based on early primary plant processes which are reflected by imagery data, but are nonvisible to the human eye or cannot be detected by RGB imaging only. The system of the present invention advantageously enables monitoring structural, color, and thermal changes of the plant and parts thereof, as well as changes of the plant or plant parts surface, for example presence of pests, particularity insects and insect eggs.

According to certain embodiments, the phenotype is selected from the group consisting of a biotic stress status including potential to develop a disease, presence of a disease; severity of a disease, a pest activity and an insect activity; an abiotic stress status including deficiency in an element or combination of elements; water stress and salinity stress; a feature predicting harvest time; a feature predicting harvest yield; a feature predicting yield quality and any combination thereof. Plant pests can include viruses, nematodes, bacteria, fungi, and insects.

According to certain embodiments, the system is further configured to generate as output data at least one of the phenotype, a quantitative phenotype, an agricultural recommendation based on said phenotype, or any combination thereof. According to these embodiments, the computing platform is further configured to deliver the output data to a remote device of at least one user.

According to certain exemplary embodiments, the agricultural recommendation relates to yield prediction, including, but not limited to, monitoring male or female organs to estimate yield, monitoring fruit maturity, monitoring fruit size and number, monitoring fruit quality, nutrient management, and determining time of harvest.

The reproducible image data obtained by the systems and methods of the present invention can be used for accurate annotation of the obtained images. Together with the vast knowledge and experience of inventors of the present invention in characterizing plant phenotypes, a plant phenotype database can be produced to be used for training an engine to detect and/or predict a phenotype of a plant.

According to additional aspect, the present invention provides a system for training an engine for detecting or predicting a phenotype of a plant, comprising:

a plurality of imaging sensors of different modalities selected from the group consisting of: a Red-Green-Blue (RGB) sensor; a multispectral sensor; a hyperspectral sensor; a depth sensor; a time-of-flight camera; a LIDAR; and a thermal sensor, the plurality of sensors mounted on a bracket at predetermined geometrical relationships;

a computing platform comprising at least one computer-readable storage medium and at least one processor for:

receiving data captured by the plurality of sensors, the data comprising at least two images of at least one part of a plant, the at least two images captured at a distance of between 0.05 m and 10 m from the plant;

preprocessing the at least two enhanced images in accordance with the predetermined geometrical relationships, to obtain unified data;

obtaining annotations for the unified data, the annotations are associated with the phenotype of the plant; and

training an engine on the unified data and the annotations, to receive images of a further plant and determine or predict a phenotype of the further plant.

According to certain embodiments, the processor is further adapted to:

receive from at least one additional sensor additional data related to positioning and/or environmental conditions of the plant; and

process the at least two images using the additional data to eliminate effects generated by the environmental conditions and/or positioning to obtain at least two enhanced images before preprocessing.

According to certain embodiments, the preprocessing comprises preprocessing the at least two enhanced images.

The engine, processor and the at least one additional sensor are as described hereinabove.

According to certain embodiments, the computing platform may also be operative in receiving information related to mutual orientation among the sensors. The computing platform may further be operative in receiving information related to mutual orientation between the sensors and at least one of an illumination source and a plant.

According to certain embodiments, training the engine is performed upon multiplicity of unified data obtained from images received at a plurality of time points.

The sensors and the system particulars are as described hereinabove.

Use of the systems and methods of the present invention is not limited to phenotyping plants, and can be used for phenotyping other objects.

Thus, according to additional aspect, the present invention provides a system of detecting or predicting a state of an object, the system comprising:

a plurality of imaging sensors of different modalities selected from the group consisting of: a Red-Green-Blue (RGB) sensor; a multispectral sensor; a hyperspectral sensor; a depth sensor; a time-of-flight camera; a LIDAR; and a thermal sensor, the plurality of sensors mounted on a bracket at predetermined geometrical relationships;

a computing platform comprising at least one computer-readable storage medium and at least one processor for:

receiving data captured by the plurality of sensors, the data comprising at least two images of at least one part of the object, the at least two images captured at a distance of between 0.05 m and 10 m from the object;

preprocessing the at least two images in accordance with the predetermined geometrical relationship, to obtain unified data;

extracting features from the unified data; and

providing the features to an engine to obtain a phenotype of said object.

According to certain embodiments, the processor is further adapted to:

receive from at least one additional sensor additional data related to positioning and/or environmental conditions of the object; and

process the at least two images using the additional data to eliminate effects generated by the environmental conditions and/or positioning to obtain at least two enhanced images before preprocessing.

According to certain embodiments, the preprocessing comprises preprocessing the at least two enhanced images.

According to certain embodiments, the computing platform may also be operative in receiving information related to mutual orientation among the sensors. According to further embodiments, the computing platform may also be operative in receiving information related to mutual orientation between the sensors and at least one of an illumination source and an object.

The sensors and the system particulars are as described herein above.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Embodiments of methods and/or devices herein may involve performing or completing selected tasks manually, automatically, or by a combination thereof. Some embodiments are implemented with the use of components that comprise hardware, software, firmware or combinations thereof. In some embodiments, some components are general-purpose components such as general purpose computers or processors. In some embodiments, some components are dedicated or custom components such as circuits, integrated circuits or software.

For example, in some embodiments, some of an embodiment may be implemented as a plurality of software instructions executed by a data processor, for example which is part of a general-purpose or custom computer. In some embodiments, the data processor or computer may comprise volatile memory for storing instructions and/or data and/or a non-volatile storage, for example a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results).

### BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 is a schematic illustration of a system, in accordance with some embodiments of the disclosure;

FIG. 2A is a flowchart of a method for training an engine for determining a phenotype of a plant, in accordance with some embodiments of the disclosure;

FIG. 2B is a flowchart of a method for preprocessing images, in accordance with some embodiments of the disclosure;

FIG. 3 is a flowchart of a method for calibrating a system for determining phenotypes, in accordance with some embodiments of the disclosure;

FIG. 4 shows exemplary images taken by different sensors and registered, in accordance with some embodiments of the disclosure;

FIG. 5 is a flowchart of a method for calibrating a thermal sensor, in accordance with some embodiments of the disclosure;

FIG. 6 is a flowchart of a method for determining a phenotype of a plant, in accordance with some embodiments of the disclosure;

FIG. 7 shows exemplary images of leaves of plants exposed to fertilizer deficiency abiotic stress taken with a variety of imaging sensors and schematic demonstration of the analyses performed for each image;

FIG. 8 demonstrates the effect of fertilizer deficiency abiotic stress on leaves;

FIG. 9 shows exemplary images of leaves of plants exposed to fertilizer deficiency abiotic stress taken with a variety of imaging sensors and schematic demonstration of the analyses performed for the multi-modal images;

FIG. 10 shows that analysis performed on the images in their totality (i.e. not cropped, as described by Grad-CAM) may focus on regions of the images that are irrelevant to the problem, such as background elements that are not plant material. This phenomenon is especially true for images taken by RGB only (FIG. 10A), vs images taken by multi-modal stack (FIG. 10B);

FIG. 11 demonstrates images obtained by the multi-modal sensors (RGB, thermal and depth sensor) and by RGB sensor only after masking and schematic presentation of the analyses performed; and

FIG. 12 demonstrates that using a mask on the relevant plant parts (leaves), results in analysis which predominantly focusses on these plant parts, and does not take into account irrelevant parts of the images, such as background features. FIG. 12A RGB only FIG. 12B multi-modal stack.

### DETAILED DESCRIPTION OF THE INVENTION

One technical problem handled by the present disclosure is the need to use manual labor for monitoring one or more plants and their environment in order to determine the best treatment of the plants or to plan for future activities, to achieve agricultural or ecocultural goals. Manual labor is costlier and less available, thus limiting its usage. Furthermore, monitoring plant phenotypes by unprofessional human labor is less accurate and reproducible, while professional human labor may be extremely expensive and not always available.

Determining the best treatment and growth conditions for a crop plant requires detection of the plant state, reflected by the plant phenotype. As used herein, the term “plant phenotype” refers to observable characteristics of the plant biophysical properties, the latter being controlled by the interaction between the genotype of the plant and its environment. The aboveground plant phenotypes may be broadly classified into three categories, including structural, physiological, and temporal. The structural phenotypes refer to the morphological attributes of the plants, whereas the physiological phenotypes are related to traits that affect plant processes regulating growth and metabolism. Structural and physiological phenotypes may be based on referring to the plant as a single object and compute its basic geometrical properties, e.g., overall height and size, or by considering individual components of the plants, for example leaves, stem, flower, fruit and further components like leaf length, chlorophyll content of each leaf, stem angle, flower size, fruit volume and the like.

The term “phenotyping” as is used herein refers to the process of quantitative characterization of the phenotype.

Earlier detection of the plant state may provide for better decisions regarding the agricultural practice to be performed and the outcome of such decisions. Such states may include, but are not limited to biotic- and a-biotic stresses that reduce yield and/or quality. It may also be useful to monitor in almost real time large areas and get fast recommendations on crop growth conditions, status of plants production and/or health and the like.

Yet another technical problem handled by the present disclosure is the need for objective decision support systems overcoming the subjectivity of manual assessment of plant status, and the recommendations based on such assessment. In determining a useful recommendation to the farmer, several components need to be considered and integrated, such as but not limited to the plant, the environment including soil, air temperature, visibility, inputs, pathogens, or the like.

The plant status is the bottom line of the mentioned factors, however, it is hardest to obtain by an automatic system since it requires, among others, high resolution and knowledge from experts. Additionally, or alternatively, the situation is dynamic as the conditions change, the plant is growing, etc., thus requiring adjustment of the system.

For a given situation, there is a need for precise recommended treatment or plans, non-subjective to knowledge gaps, different conceptions, or the like.

One technical solution is a system comprising a multiplicity of sensors, and in particular imaging sensors, for capturing one or more parts of one or more plants. The sensors may include one or more of the following: RGB sensor, a multi spectral sensor, a hyperspectral sensor, a thermal sensor, a depth sensor such as but not limited to a LIDAR or a stereo vision camera, or others. The sensors are mounted on a bracket in predetermined geometrical relationship. The system may also comprise additional sensors, such as a radiation sensor, a temperature sensor, a humidity sensor, a position sensor, or the like. It will be appreciated that data from sensors of one or more types may be useful for processing images captured by a sensor of another type. For example, a light intensity sensor may be required in order to process images taken by a multispectral or hyperspectral sensors.

As used herein, the term “a plurality” or “multiplicity” refers to “at least two”.

According to certain embodiments, the RGB camera is selected from the group consisting of an automatic camera and a manual camera.

Each sensor may be calibrated individually, for example under laboratory conditions. The sensors may then be installed on the bracket, and the system may be calibrated as a whole, to adjust for the different fields of view and to eliminate changing conditions within the system and environmental conditions. In some embodiments, calibrating one or more sensors may be performed after installation on the bracket.

In some embodiments, decision mechanisms may be used, in particular neural networks, for which a training phase may take place, in which a multiplicity of images captured by different modalities may be acquired by the imaging sensors. The images may be captured consecutively or sporadically. The images may be pre-processed, possibly including the fusion of data received from the additional sensors, to eliminate noises and compensate for the differences in the sensors and their calibration parameters, the environment, and measurement errors, and to improve sensors signals (e.g. by improving their resolution).

The images may then be registered, for example one or more of the images may be transformed such that the images or parts thereof correspond and the locations of various objects appearing in multiple images are matched, to generate a unified image.

One or more captured images, or the unified image may be annotated, also referred to as labeled by a user, to indicate a status, a prediction, or recommendation, or the like.

Features may then be extracted from the unified image, using image analysis algorithms.

The features and annotations may then be used for training an artificial intelligence engine, such as a neural network, a deep neural network, or the like.

A runtime phase may then take place, in which images of the same nature as used during the training phase are available. During runtime, the images of various plants or plant parts may be preprocessed and registered as on the training phase, and features may then be extracted.

The features and optionally additional data as acquired directly or indirectly from the additional sensors, may be fed into the trained engine, to obtain a corresponding status, prediction or recommendation.

It will be appreciated that the engine may be trained upon data collected from a multiplicity of systems, to provide for more sets of various conditions, possible various imaging sensors, or the like.

It will be appreciated that a trained engine may be created per each plant type, per each plant type and location, or for multiple plant types and possibly multiple locations, wherein the plant type or the location may be indicated as a feature. Additionally, or alternatively, the engines may be obtained from other sources.

It will be appreciated that a trained engine may be created per each type of a plant disease, per each type and the severity of a plant disease. Alternatively, a trained engine may be created for multiple plant species and plant crops, multiple plant diseases and possibly multiple grades of disease severity, wherein the type of plant disease or the disease severity may be indicated as a feature.

However, it will be appreciated that other decision engines, which do not require training may be used. Such engines may include but are not limited to rule-based engines, Look up table (LUT), or the like.

The system may be adapted for taking images from a relatively short distance, for example, between about 5 cm and about 10 m, between about 5 cm and about 5 m, or between about 50 cm and about 3 m, or between about 75 cm and about 2 m, or the like. In such distances, the difference in the viewing angles between different imaging sensors mounted on the bracket may be significant and cannot be neglected. Also, due to the small size of the captured items for example leaves or parts thereof, the resolution of the used sensors may be high, for example better than 1 mm.

In some embodiments, the system may be designed to be carried by a human, mounted on a car or on an agricultural vehicle, carried by a drone, or the like. In further embodiments, the system may be installed on a drone or another flying object, or the like.

In some embodiments, the system may be designed to be mounted on a cellular phone.

One technical effect of the disclosure is the provisioning of a system for automatic determination of phenotypes of plants, such as but not limited to: yield components, yield prediction, properties of the plants, biotic stress, a-biotic stress, harvest time and any combination thereof.

Another technical effect of the disclosure is the option to train such system to any plant and also to objects other than plants, at any environment, wherein the system can be used in any manner—carried by a human operator, installed on a vehicle, installed on a flying device such as a drone, or the like. Training on any such conditions provides for using the system to determine phenotypes or predictions from images captured with corresponding conditions.

Yet another technical effect of the disclosure is the option to train the system for certain plant types and conditions in one location, and reuse it in a multiplicity of other locations, by other growers.

Yet another technical effect of the disclosure is the provisioning of quantitative, reproducible results, in a consistent manner.

Yet another technical effect of the disclosure is the ability to detect the plant status and integrate it with additional aspects (e.g. environment, soil, etc.) to produce useful recommendation to the farmers.

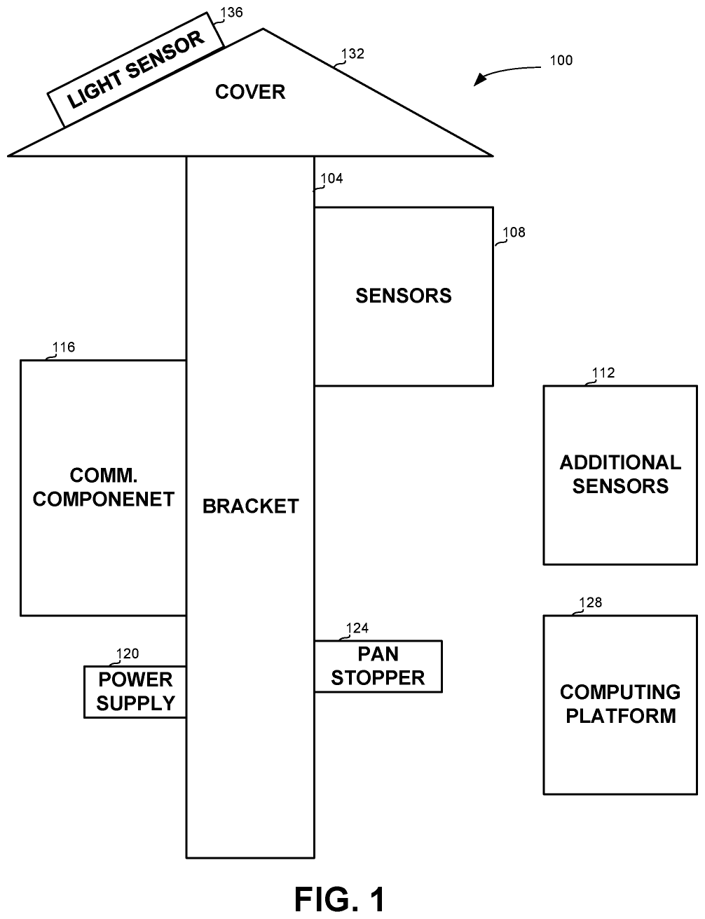

Referring now to FIG. 1, showing a schematic illustration of a system, in accordance with some embodiments of the disclosure.

The system, generally referenced 100, comprises a bracket 104 such as a gimbal. Bracket 104 may comprise a pan stopper 124 for limiting the panning angle of the gimbal.

A plurality of sensors 108 may be mounted on the gimbal. Sensors 108 may be mounted separately or as a pre-assembled single unit. The geometric relations among sensors 108 are predetermined, and may be planned to accommodate their types and capturing distances, to make sure none of them blocks the field of view of the other, or the like.

Sensors 108 may comprise a multiplicity of different imaging sensors, each of which may be selected among an RGB camera, a multi spectral camera imaging at various wave lengths, a depth camera and a thermal camera. Each of these sensors is further detailed below.

System 100 may further comprise a power source 120, for example one or more batteries, one or more rechargeable batteries, solar cells, or the like.

System 100 may further comprise at least one computing platform 128, comprising at least a processor and a memory unit. Computing platform 128 may also be mounted on bracket 100, or be remote. In some embodiments, system 100 may comprise one or more collocated computing platforms and one or more remote computing platforms. In some embodiments, computing platform 128 can be implemented as a mobile phone mounted on bracket 104.

System 100 may further comprise communication component 116, for communicating with a computing platform 128. If computing platform 128 comprises components mounted on bracket 104, communication component 116 can include a bus, while if computing platform 128 comprises remote components, communication component 116 can operate using any wired or wireless communication protocol such as Wi-Fi, cellular, or the like.

System 100 can comprise additional sensors 112, such as but not limited to any one or more of the following: a temperature sensor, a humidity sensor, a position sensor, a radiation sensor, or the like. Some of additional sensors 112 may be positioned on bracket 104, while others may be located at a remote location and provide information via communication component 116. Additional sensors 112 may be mounted on bracket 104 at predetermined geometrical relationships with plurality of sensors 108. Predetermined geometrical relationships may relate to planned and known locations of the sensors relatively to each other, comprising known translation and main axes rotation, wherein the locations are selected such that the fields of view of the various sensors at least partially overlap.

Imaging sensors 108 may comprise an RGB sensor. The RGB sensor may operate at high resolution, required for measuring geometrical features of plants, which may have to be performed at a level of a few pixels. In an exemplary embodiment, a BFS-U3-2006SC camera by Flir® may be used, having a resolution of 3648×5472 pixels, pixel size of 2.4 μm, sampling rate of 18 frames per second, and weight of 36 g, with a lens having a focal length of 16 mm. Such optical properties provide for a field of view of 30°×45°, with angular resolution of 0.009°. This implies that within a range of 1 m, one pixel covers size of 0.15 mm.

Imaging sensors 108 may comprise a multi spectral sensor, having a multiplicity of narrow bandwidths. For example, the multi spectral sensor may operate with 7 channels of 10 nm full width at half maximum (FWHM), as shown in Table 1 below.

TABLE 1
Multi Spectral Sensor Operation

| | | Center Line | FWHM* |
|---|---|---|---|
| Band No. | Band Name | (mm) | (mm) |
| 1 | Blue | 480 | 10 |
| 2 | Green Edge | 520 | 10 |
| 3 | Green | 550 | 10 |
| 4 | Red | 670 | 10 |
| 5 | Red Edge 1 | 700 | 10 |
| 6 | Red Edge 2 | 730 | 10 |
| 7 | Near Infra-Red | 780 | 10 |
| 8 | RGB | 450/550/650 | 100 |

The multi spectral sensor may be operative in determining properties required for evaluating biotic and a-biotic stress of plants. For example, the channels in the green and blue areas provide for assessing the chlorophyll a and anthocyanin: chlorophyll a is characterized by absorbing the blue area, and the gradient formed by two wavelengths in the green area provides measuring of pigments. The red channel provides data for measuring chlorophyll b. The red and near infra-red channels may be located on the red edge, which provides a means for measuring changes in the geometric properties of the cells in the spongy mesophyll layer of the leaves and other general stress in the plant.

In an exemplary embodiment, a multi spectral camera by Sensilize® may be used, having a resolution of 640×480 pixels, weight of 36 g, with a lens having a focal length of 6 mm. These properties provide for a field of view of 35°×27°, with angular resolution of 0.06°. This implies that within a range of 1 m, one pixel covers size of 1 mm.

Imaging sensors 108 may comprise a depth sensor, operative for: complementing the data obtained by the RGB camera, such as differentiating between plant parts, with geometrical dimensions, thus enabling the measurement of geometrical sizes in true scale; and depicting the three-dimensional structure of plants for radiometric correction of the multi spectral sensor and the thermal sensor. The depth camera may provide an image of 1 mm resolution at lm distance and depth accuracy of 1 cm. However, as more advanced sensors become available, better performance can be achieved. Additionally, or alternatively, a depth map may be created by a time of flight camera, by a LIDAR, using image flow techniques, or the like.

Imaging sensors 108 may comprise a thermal camera for measuring the temperature of plant parts, such as leaves. The leaves temperature may provide an indication to the water status of the plant. Additionally, or alternatively, the temperature distribution over the leaves may provide indication for leaf injuries or lesions due to the presence of diseases or pests.

In an exemplary embodiment, a Therm-App camera by Opgal® (Haifa, Israel), may be used, having a resolution of 384×288 pixels, pixel size of 17 μm, sampling rate of 12 frames per second, and weight of 100 g, with a lens having a focal length of 13 mm. Such optical properties provide for a field of view of 30°×22°, with angular resolution of 0.08°. This implies that within a range of 1 m, one pixel covers size of 1.3 mm

It will be appreciated that in addition to data obtained by combining information from a multiplicity of sensors as detailed below, some data may also be obtained from a single or multiple imaging sensors. For example:

Analyzing a single thermal image for temperature distribution within the image provides for identifying relative stress, e.g., local anomaly on distinct leaves or plants, which may provide an early indication of a stress. The temperature differences which indicate such stresses are of the order of few degrees. Thus, a thermal camera with sensitivity of about 0.5° provides for detecting these differences. However, as more advanced sensors become available, higher sensitivity can be achieved.

In order to detect absolute stress, and provide quantitative measure thereof, it may be required to normalize the leaf temperature in accordance with the environmental temperature, relative humidity, radiation and wind speed, which may be measured by other sensors. The differences required to be measured in the leaf temperature depend on the plant water status. Higher accuracy provides for differentiating smaller differences in the water status of plants. For example, accuracy of 1.50 degrees Celsius has been proven sufficient for assessing the water state of grapevine and cotton plants.

Combinations of images from different imaging sensors may provide for various observations, for example:

A combination of an RGB, multi-spectral sensors or thermal sensors and optionally external lighting monitoring can provide for early detection of stresses before symptoms are visible to the human eye or by RGB images. According to certain exemplary embodiments, the combination of an RGB, multi-spectral sensors or thermal sensors and optionally external lighting monitoring can provide for early detection of stress caused by fertilizer deficiency. Additionally, or alternatively, combination of an RGB, thermal and depth sensors can provide for early detection of stress caused by fertilizer deficiency.

A combination of multi-spectral and lighting sensors can provide for identifying significant signature differences between healthy and stress plants.

An RGB sensor can provide for distinguishing between plant parts. An RGB sensor may thus provide for detecting changes in leaf color, a depth sensor may provide for detecting changes in plant size and growth rate; a thermal sensor may provide for detecting changes in transpiration. Combinations of the above can provide for early detection of lack of water and early detection of lack of fertilizer.

According to certain embodiments, the combination of imagining sensors comprises RGB sensor, multispectral sensor, depth sensor and thermal sensor.

It will be appreciated that one or more sensors may have different roles in the detection at different stages of the growth cycle.

Additional sensors 112 may include an inertial sensor for monitoring and recording of optical head direction, which may be useful in calculating the light reflection from plant organs, independent of the experimental conditions. An exemplary inertial sensor is VMU931 having a total of nine axes for gyro, accelerometer and magnetometer, and equipped with calibration software. The inertial sensor may also be useful in assessing the motion between consecutive images, and thus evaluate the precise position of the system, for calculating the depth map and compensating for the smearing effects caused due to motion.

Additional sensors 112 may include other sensors, such as temperature, humidity, location, or the like.

All sensors mounted on bracket 104 may be controlled by a command and control unit, which may be implemented on computing platform 128 or a different platform. The command and control unit may be implemented as a software or hardware unit, responsible for activating the mounted sensors, with adequate parameter setting, which may depend, for example on the plant, the location, the required phenotypes, or the like. The command and control unit may be further operative to perform any one or more of the following actions: setting a parameter of a sensor from the plurality of sensors; operating the processor in accordance with a selected application; providing an indication to an activity status of a sensor; providing an indication to a calibration status of a sensor; and recommending to a user to calibrate. The command and control unit may also be operative in initiating the preprocessing of the images, registering the images, providing the images or features thereof to the trained engine, providing the results to a user, or the like.

It will be appreciated that the sensors may operate under different operating systems such as Windows®, Linux®, Android® or others, use different communication protocols, or the like. Computing platform 128 may be operative in communicating with all sensors, receiving images and other data therefrom, and continuing processing the images and data.

In some embodiments, system 100 may optionally comprise a cover 132, and one or more light intensity sensors 136 positioned on cover 132. Light intensity sensors 136, which may measure ambient light intensity in predefined wavelength bands, may be used to reduce the effect of different background light created by the differences in weather, clouds, time of day, or the like. The light sensors may be used for the normalization of the images taken by multi-spectral and RGB cameras or by other optical sensors.

In some embodiments, system 100 may comprise a calibration target for recording the light conditions by one or more sensors, for example sensors 108 comprising an additional set of the similar sensors. The calibration target may be a permanently mounted target or a target that performs a motion to appear in the field of view.

In some embodiments, system 100 may be implemented as a relatively small device, such as a mobile phone or a small tablet computer, equipped with a plurality of capture devices, such as an RGB camera and a depth, thermal, hyperspectral or multispectral camera, with or without additional components such as cover 132 or others. The various sensors may be located on the mobile phone with predetermined geometrical relationship therebetween. Such device may already comprise processing, command and control, or communication capabilities and may thus require relatively little or no additions.

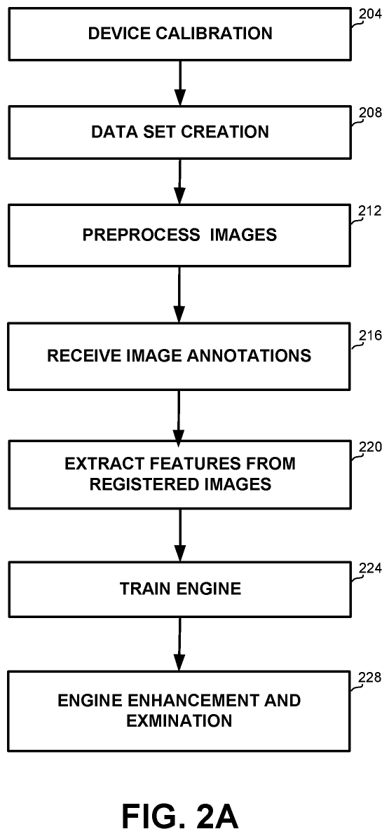

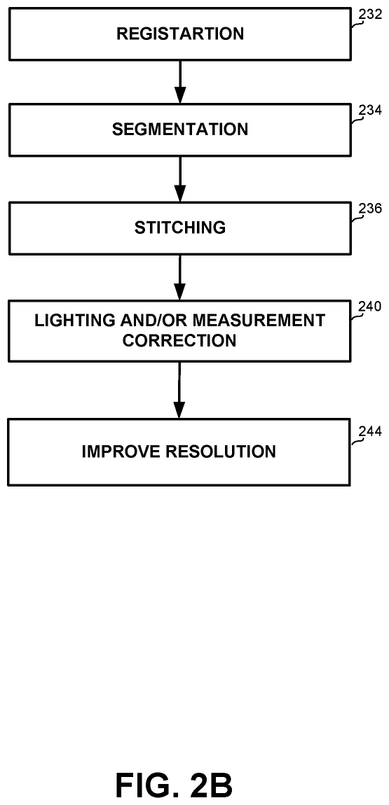

Referring now to FIG. 2A, showing a method for training an engine for determining a phenotype of a plant based on images taken by a system, such as the system of FIG. 1.

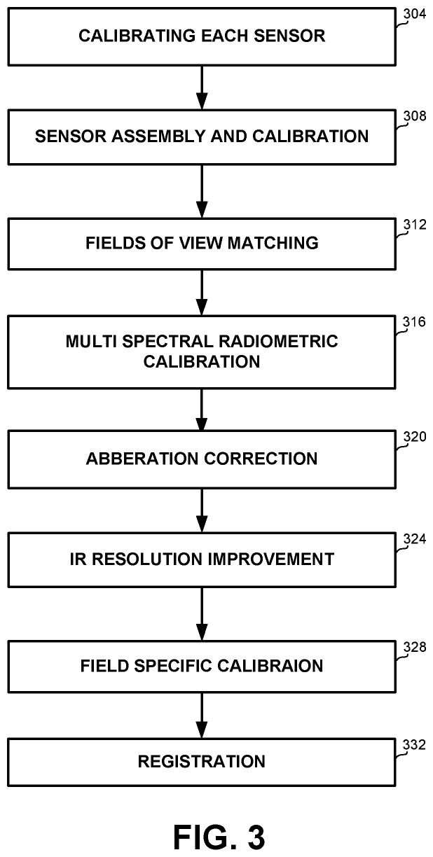

Before training the engine may begin, the system needs to be assembled and calibrated, as detailed in association with FIGS. 3-5 below.

On step 204, the device may be calibrated, in order to match the parameters of all sensors with their locations, and possibly with each other. The device calibration is further detailed in association with FIG. 3 below.

On step 208, a data set may be created, the data set comprising a multiplicity of images collected from the various imaging sensors 108, as described in association with

FIG. 1 above and as calibrated in accordance with FIG. 3 below. The data set may also comprise data received from additional sensors 112. Each image or image set may thus be associated with information related to the parameters under which it was taken, and additional data, such as location, position, e.g., which direction the imaging sensor is facing, environmental temperature, plant type, soil data, or the like. On step 212, the images may be preprocessed to eliminate various effects and enhance resolution, and may be registered. Preprocessing is further detailed in association with FIG. 2B below.

On step 216 one or more annotations may be received for each captured image or registered image, for example from a human operator. The annotations may include observations related to specific part of a plant, size of an organ, color of an organ, a state of the plant, such as stress of any kind, pest, treatment, treatment recommendation, observation related to the soil, the plot, or the like. In some embodiments, the process of FIG. 2 may be performed retroactively, such that annotations reflect actual information which was not available at the time the images have been taken, such as yield.

On step 220, features may be extracted from one or more of the captured or registered images. The features may relate to optical characteristics of the image, to objects identified within the images, or others. The registration enables to extract features obtained from one or more sensors. For example, once RGB and thermal images are registered, valuable leaf temperature data can be achieved, which could not be achieved without the registration.

On step 224, the extracted features, and optionally additional data as received from additional sensors 112, along with the provided annotations may be used to train an artificial intelligence engine, such as a neural network (NN), a deep neural network (DNN), or others. Parameters of the engine, such as the number of layers in a NN, may be determined in accordance with the available images and data. The engine may be retrained as additional images, data, and annotations are received. The engine training may also include testing, feedback and validation phases.

In some embodiments, a separate engine may be created for each type of object, particularly of a plant, each location/plot, geographical area, or the like. In other embodiments, one engine may serve a multiplicity of plant types, plots, geographical areas, or others, wherein the specific plant type, plot, or geographical area are provided as features which may be extracted from the additional data.

On step 228 the engine may be tested by a user and enhanced, for example by adding additional data, changing the engine parameters, or the like. Testing may include operating the engine on some images upon which it was trained, or additional images, and checking the percentage of the responses which correspond to the human-provided labels.

In some embodiments, the provided results may be examined, by the engine providing an indication which area of the unified image or the images as captured demonstrates the differentiating factor that caused the recognition of the phenotype. The indication may be translated to a graphic indication displayed over an image on a display device, such as a display of a mobile phone.

Referring now to FIG. 2B, showing a flowchart of steps in a method for preprocessing the images.

Preprocessing may include registration step 232. Registration may provide for images taken by imaging sensors of different types to match, such that an object, object part, or feature thereof depicted in multiple images is identified cross-image. Registration may thus comprise alignment of the images.

In particular, registration provides for fusing information from different sensors to obtain information in a number of ranges, including visible light, Infra-Red, and multispectral ranges in-between.

In order to register images without manual indication of points of interest, information from the depth camera, which provides information of the distance between the system and the captured objects may be used to register RGB images, thermal images and multi spectral images, using also the optical structure of the system and the geometric transformation between the cameras.

Thus, when registering images, a depth image may be loaded to memory, and the distance to a depicted object, such as a leaf, fruit, or stalk is evaluated. Further images, for example RGB or multi spectral images, may then be loaded, and using the geometric transformations between the cameras, each such image is transformed accordingly.

Following the transformations, the images can be matched, and features may be extracted from their unification.

Additionally, or alternatively, registration may be performed by other methods, such as deep learning, or a combination of two or more methods.

Preprocessing may include segmentation step 234, in which one or more images or parts thereof are split into smaller parts, for example parts that depict a certain organ.

Preprocessing may include stitching step 236, in which two or more images or parts thereof are connected, i.e., each contributes pats or features not included in others, for creating a larger image, for example of a plot comprising multiple plants.

Preprocessing may include lighting and/or measurement correction step 240. Step 240 may comprise radiometric correction, which may include: 1. correcting the target geometry, which relates to the surface evenness, pigment, humidity level and the unique reflection spectrum of the material of the captured image; 2. correcting errors caused by the atmosphere between the sensor and the captured object, including particles, aerosols and gases; and 3. physical geometry of the system and the captured object, also known as Bidirectional reflectance distribution function (BRDF). In some embodiments, the BRFD may be calculated according to any known model or a model that will be developed in the future, such as the Cook-Torrence model, the GGX model, or the like.

Correction step 240 may include geometric transformations between images captured by different modalities, such as translation, scaling and rotations between images, for correcting the measuring angles, aspects of 3 dimensional view, or the like.

Preprocessing may include resolution improvement step 244, for improving the resolution of one or more of the images, for example images captured by a thermal sensor, a multi spectral camera, or the like.

Referring now to FIG. 3, showing a flowchart of a method for calibrating a system for determining phenotypes, in accordance with some embodiments of the disclosure. Due to a possible usage of the system in identifying plant phenotypes, the system needs to be calibrated to relevant sizes as detailed above, for example a capture distance of between 0.1 m and 10 m such as about 1 m, and high resolution, for example better than 1 mm.

On step 304 each sensor may be calibrated individually for example by its manufacturer, in a lab, or the like. During calibration, parameters such as resolution, exposure times, frame rate or others may be set. These parameters may be varied during the image capturing and the image may be normalized in accordance to the updated capturing parameters.

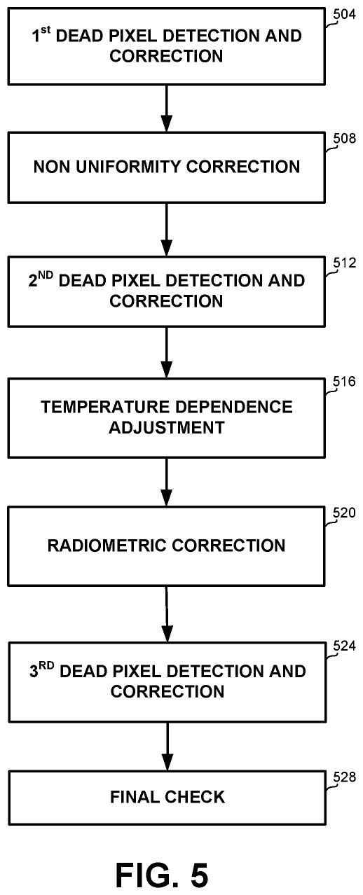

An exemplary method for calibrating a thermal sensor is detailed in FIG. 5 and the associated description below.

On step 308, the imaging sensors may be assembled on the bracket and calibrated as a whole.

The mutual orientation among the sensors, and between the sensors, an illumination source or a plant may be determined or obtained, and utilized.

The calibration process is thus useful in obtaining reliable physical output from all sensors, which is required for thermal and spectral analysis of the output. Due to the high variability of the different sensors, lab calibration may be insufficient, and field calibration may be required as well.

On step 312 the fields of view of the various sensors may be matched automatically, manually or a by combination thereof. For example, initial matching may be performed by a human, followed by finer automatic matching using image analysis techniques.

On step 316, radiometric calibration may be performed, for neutralizing the effect of the different offsets and gains associated with each pixel of each sensor, and associate physical measures to each, expressed for example as Watt/Steradian.

The radiometric calibration is thus required for extracting reliable spectral information from the imaged objects or imaged scene, such as reflectance values in a reflective range, or emission values in a thermal range. The extraction of unique thermal signature of an object is affected by: a. various noises and disturbances; optical distortions; c. atmospheric disturbances and changes in the spectral composition of the environmental illumination; and d. the reflection and emission of radiation from the imaged object, which depends also on its environment. Thus, some aspects of the sensors may be calibrated during the calibration of each sensor on step 304, while other aspects are handled when calibrating the device as a whole.

It will be appreciated that RGB cameras and depth cameras are mainly used for extracting geometric information, thus radiometric calibration of these cameras may not be necessary.

As for other sensors, including multi spectral sensors and thermal sensors, the system noise and optical distortions may be handled as part of the lab calibration. However, the atmospheric disturbances and reflections need to be handled at the capturing time and location, since the spectral composition of the light, as well as the geometrics of the depicted objects differ between the lab and the field at which the sensor is used. It will be appreciated that correcting errors stemming from the geometry of the objects is enabled by the availability of geometric information provided by the RGB, depth and position sensors included in the system. Such sensors may provide data such as the shape of the depicted object, the angle of the depicted objects relative to the camera, or the like.

Similar to the multi spectral system, the thermal sensor is highly affected by changes between the conditions in the lab and in the field. Factor (d) above, i.e., the reflection and emission of radiation from the imaged object has impact on the temperature measurement of a surface. In some situations, large angles between the perpendicular to the depicted surface and the optical axes of the plant can cause errors in the temperature measurement. However, the availability of geometric information provides for correcting such errors and more accurately assessing the leaf temperature and evaluating biotic and abiotic stress conditions.

Correcting the distortions and disturbances provides for receiving radiometric information from the system, as related to spectral reflection in each channel. Such information provides reliable base for analysis and retrieval of required phenotypes, such as biotic and abiotic stress using spectral indices or other mathematical analyses.

On step 320, distortion aberration correction may be performed. This aberration can be defined as a departure of the performance of an optical system from the predictions of paraxial optics. Aberration correction is thus required to eliminate this effect. This correction may be done, for example, by standard approaches involving chess board for the distortion correction.

On step 324, IR resolution improvement may take place in order to improve the resolution of the thermal sensor.

On step 328, field specific calibration may take place, in which parameters of the various sensors or their relative positioning may be enhanced, to correspond to the specific conditions at the field where the device is to be used. This calibration is aimed at eliminating the effect of the changing mutual orientation between the light source, the looing direction of the sensor, and the normal to the capturing plane. The calibration may determine an appropriate bidirectional reflectance distribution function (BRDF).

In order to use data and image gathered in different environmental conditions, the exposure time and amplification of the sensors should be adjusted to the lighting conditions. In order for the data to be independent of the variations of these parameters, the captured images need to be normalized, for example in accordance with the following formula:

$${I ( λ )}_{i , j} = {P ( λ )}_{i , j} \frac{{{I}_{0} ( λ )}_{i , j} - {D ( λ )}_{i , j}}{T ( λ ) \cdot G ( λ )}$$

### Wherein:

D(λ)_{i,j} is the dark value of the specific multi spectral (MS) waveband λ of pixel (I, J), in gl units
I(λ)_{i,j} is the value of the specific MS waveband λ after calibration on pixel (i,j) in gl units
I₀(λ)_{i,j} is the value of the specific MS waveband λ measured in front of the integration sphere on pixel (i,j), in Watt/steradian units
T(λ) is the integration time of the specific MS waveband λ, in mSec units
G(λ) is the gain of the specific MS waveband λ, in dB units
P(λ) is the radiometric calibration factor value of the corresponding band correcting the vignetting and supplying the physical units, in Watt/steradian units.

The calibration output is thus a system comprising a plurality of imaging sensors, which can be operated in any environment, under any conditions and in any range. The product of the radiometric and geometric correction provides for normalizing image values, in order to create a uniform basis for spectral signatures complying with the following rule: radiation hitting an object is converted into reflected radiation, transferred radiation, or absorbed radiation, in each wavelength separately.

On step 332, registration of the images taken by the various sensors and normalized may be performed.

Registration may comprise masking, in which the background of the RGB image is eliminated, such that an object of interest, for examples leaves is distinguished. Once registration is complete all images are aligned, and the background of the other images may be eliminated in accordance with the same mask. The data relevant to the leaves or other parts of the plant can then be extracted.

The registration process may use any currently known algorithm, or any algorithm that will be known in the future, such as Feature based (surf or sift) registration, Ransac, Intensity based, cross-correlation, or the like.

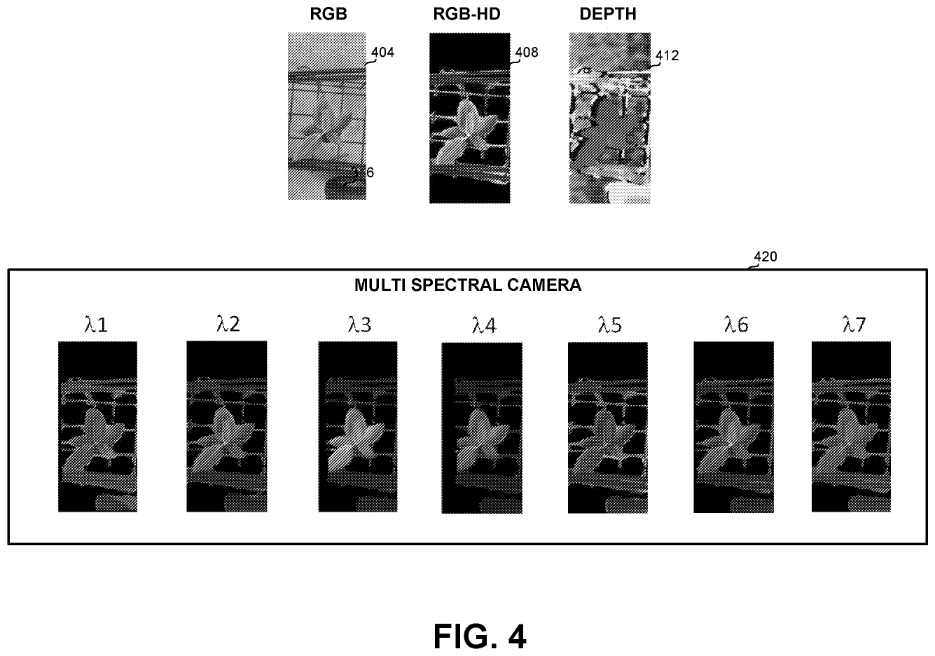

Referring now to FIG. 4, showing examplary images taken by different sensors, following their registrations. All images have been taken form a distance of about lm and at a resolution that enables differentiating details smaller than 1 mm this relates to the specific images.

Thus, RGB image 404 was taken by an RGB camera in wave length as detailed on band number 8 in Table 1 above, RGB-HD image 408 was taken by a high definition RGB camera, depth image 412 was taken by a depth camera, such that the color of each region indicates the distance between the camera and the relevant detail in the image, and multi spectral images 420 show the images taken in seven wave lengths, as detailed in bands number 1-7 of Table 1 above. It is seen that all images show the same details of the plant and its environment at the same size and location, thus enabling combining the images. The registration thus compensates for the different scales, paralax, and fields of view of the various sensors.

Referring now to FIG. 5, showing a flowchart of an exemplary method for calibrating a thermal sensor, in accordance with some embodiments of the disclosure.

On step 504, a first detection and correction of dead pixels may be performed. Dead pixels are pixels for which at least one of the following condition holds: no response to temperature changes; initial voltage higher than offset voltage; pixels whose sensitivity deviates from the sensitivity of the sensor in more than predetermined threshold, for example 10%; and pixels having noise level exceeding in at least a predetermined threshold the average noise level of the sensor, for example 50%. The correction of the dead pixels may be performed automatically by software.

On step 508, non-uniformity correction may be performed, for bringing all pixels into a unified calibration curve, such that their reaction to energy changes is uniform. The reaction of the sensor depends on the internal temperature and on the environmental temperature. In order to reduce the complexity, a linear model is created for each of these parameters separately.

On step 512, a second detection and correction of dead pixels may be performed, as detailed in association with step 504 above.

On step 516, environmental temperature dependence adjustment may be performed. The environmental temperature may be measured by a number of sensors located on the thermal sensor and the optical components. One or more matrices may be determined, defining the relationship between the different measured temperatures and the energy level measured by the sensor. This relationship may then be fitted to a polynomic, for example of third or higher degree.

On step 520, radiometric correction may be performed. At this step, the energy measured by the thermal sensor is calculated as temperature

On step 524, a third detection and correction of dead pixels may be performed, as detailed in association with step 504 above.

Further calibration of thermal images to a dimensionless stress index may be done. For example, Crop Water Stress Index (CWSI), which is defined as CWSI=(T_{leaf}−Tₘᵢₙ)/(Tₘₐₓ−Tₘᵢₙ), where T_{leaf} is the leaf temperature as measured by the thermal sensor, Tₘᵢₙ is the lower reference temperature of a completely non-stressed leaf at the same environmental conditions, and Tₘₐₓ is the upper reference temperature of a completely stressed leaf at the same environmental conditions. Tₘᵢₙ and Tₘₐₓ are either empirically estimated, or practically measured at the same scene where the leaves are measured, or theoretically calculated from energy balance equations.

On step 528, a final check may be performed.

In some embodiments, the first and second dead pixel corrections are performed automatically, while the third correction is performed manually.

It will be appreciated that the flowcharts of FIGS. 3 and 5 and the associated descriptions are exemplary only, and may change in accordance with the types of used sensors, the specific sensor models used, varying conditions, professional preferences and considerations, or the like.

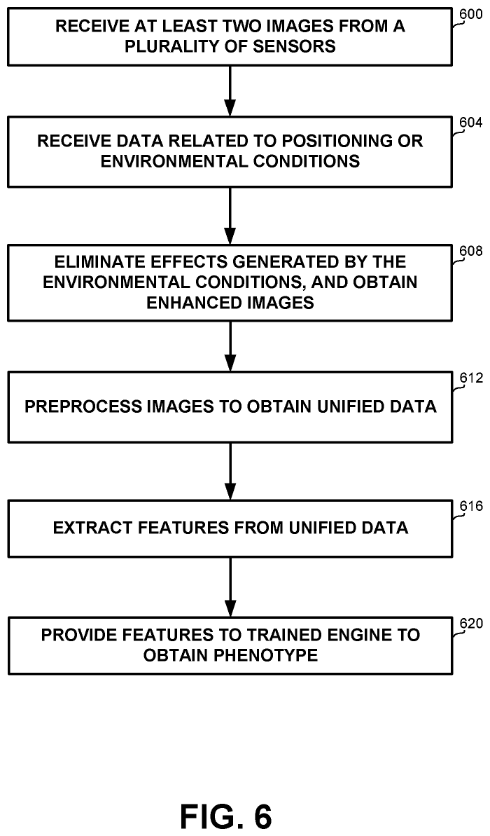

Referring now to FIG. 6, showing a flowchart of a method for determining a phenotype of a plant, in accordance with some embodiments of the disclosure. The method may be performed once the system is assembled and calibrated, and an engine has been trained with relevant data, comprising for example data related to the same plant, plot, or other characteristic.

In some embodiments, at least some steps of the method of FIG. 6 may be performed by computing platform 128 of FIG. 1. In some embodiments, some of the steps may be performed by computing platform mounted on bracket 104, and other steps may be performed by one or more computing platforms located remotely from bracket 104, such as a cloud computer. In further embodiments, all processing may be performed by a cloud computer, which may receive the data and return the processing result to the computing platform mounted on bracket 104. The steps may be performed by executing one or more units, such as an executable, a static library, a dynamic library, a function, a method, or the like.

On step 600, at least two images may be received from at least two imaging sensors of different types, from plurality of sensors 108 mounted on bracket 104 of system 100. Each image may be an RGB image, a multi spectral image, a depth image, a thermal image, or the like.

On step 604, data related to positioning of system 100 may be received from additional sensors 112. The data may be received from additional sensors mounted on bracket 104, additional sensors located remote from bracket 104, or a combination thereof.

On step 608, the at least two images may undergo elimination of effects generated by the environmental conditions to obtain enhanced images, using the data obtained from the additional sensors. The mutual orientation between imagers, illumination source and plant, may also be used. Preprocessing may use calibration parameters obtained during system calibration, and corrections determined as detailed for example in association with step 212 above, such that the images are normalized.

On step 612 the enhanced images may be preprocessed, as described for example on FIG. 2B above. Preprocessing may include registration, in which the images are aligned, such that areas in two or more images representing the same objects or parts thereof appearing in two or more images are matched. The registration provides for unified data, whether in the format of a unified image or in any other representation.

On step 616, one or more features may be extracted from the unified data. The features may be optical features, plant-related features, environment-related features, or the like. The features may be extracted using image analysis algorithms.

On step 620, the extracted features and optionally data items from the additional data may be provided to an engine, to obtain a phenotype of the plant, thus using the multi-dimensional sensor input to quantify or predict disease and/or stress level based on multi-modal data.

The engine may be a trained artificial intelligence engine, such as a neural network or a deep neural network, but may also be a non-trained engine, such as a rule engine, a look up table, or the like. In some embodiments, a combination of one or more engines may be used for determining a phenotype based on the features as extracted from the multi modal sensors. pre-trained models and non-trained models as a starting point in a network adapted to the analysis of data from a plurality of multi-modal sensors. The phenotype can be provided to a user or to another system using any Input/Output device, written to a file, transmitted to another computing platform, or the like.

In some embodiments, the results provided by the engine may be examined using class activation map. For example, the engine may provide an indication to which area of the unified image or the images as captured demonstrates the differentiating factor that caused the recognition of the phenotype. The indication may be translated to a graphic indication displayed over an image on a display device, such as a display of a mobile phone. Thus, the degree of overlap between the internal neural network representation and the segmented objects is presented and can be useful in evaluating the degree of success the neural network, and in guiding a neural network towards phenotypically relevant regions of the plant using the well-aligned multi-layer data as a basis.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Early Detection of Abiotic Stress—Different Imaging Sensors and Combinations Thereof

Symptoms of abiotic stress were used to assess the effect of combination of a plurality of imaging sensors of different modalities on early detection. The biological system used was leaves of banana plantlets induced for abiotic stress by deficient fertilizer application.

One-month old Banana plantlets were grown in a 1 L pot in a commercial greenhouse. 51 plants were watered and fertilized every day according to the normal commercial growing conditions (100% fertilized, no induction of stress), and 51 plants were watered every day with the same amount of water but without fertilizer (0% fertilized, maximum stress). The experiment was conducted for 52 days, and images were collected at different 32 days using the system of the invention, including Red-Green-Blue (RGB), multi-spectral sensor, depth and thermal camera as detailed below (defined as “AgriEye”). The cameras were connected to a tripod and all the cameras were facing down (90 degrees to the tripod). All images were taken at a distance of 1 meter from the plants. The operator moved the tripod with the AgriEye set of camera from plant to plant and using collected the data from all the sensors using a tablet. Data collection time was between 07:00 AM and 10:00 AM. Watering of the plants with or without fertilizer was at 13:00. All the collected data was uploaded to a database.

Early detection of stress is defined as detection prior to the symptoms being visible in an image captured by an RGB camera. Late stress detection is defined as the symptoms being visible in an RGB image, e.g., a visible difference in plant size (height and leaf number).

The following sensors were used:
RGB camera, 3FS-U3-200S6C-C (Flir Systems, Inc.)
Multi-spectral sensor, 7 channel multispectral camera RobinEye (Sensilize)
Thermal camera, ThermApp (Opgal)
Depth camera, RealSense (Intel)

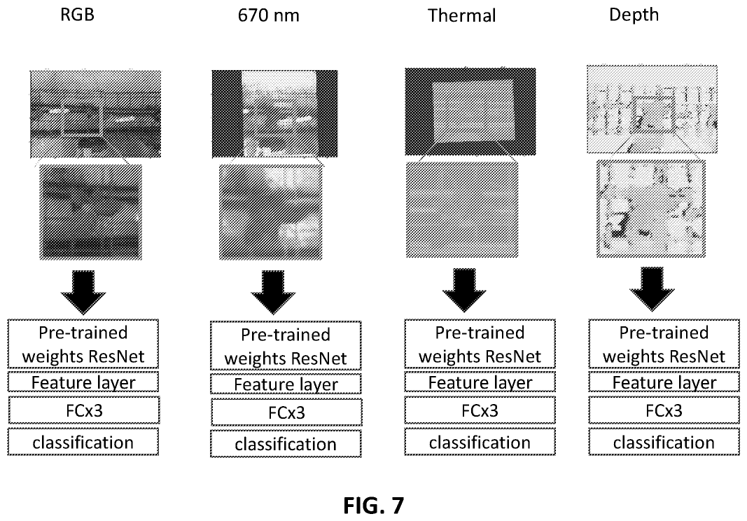

FIG. 7 shows exemplary images of leaves taken by each of the sensors. As can be taken from the upper panel, the plants are small, and thus end-to-end capture is not effective. Determination of regions of interest (ROI) is required, as described, for example, in Vit y et al., 2019 (Vit A et al. The IEEE Conference on Computer Vision and Pattern Recognition (CVPR) Workshops, Jun. 16-20 2019). The middle panel of FIG. 7 shows the ROI of the pictures taken by each of the imaging sensors. The images of the ROI were analyzed by making use of a deep neural network (ResNet50), with weights pre-trained on a classification task from the ImageNet database. The feature layer of the network was coupled to a 3-layer deep fully-connected neural network with output the relevant classes, as is customary for a transfer learning scheme. Based on this analysis, the images were classified as “stressed” or “unstressed”, referring to abiotic stress. The ground-truth for the multi-modal data was set by the description of the treatment known (stress vs. no stress). Accuracy is defined as the percentage of correct classifications at the early stage out of the total number of cases.

Table 2 below shows the accuracy (correct detections divided by the total number of detections) for analysis using the various sensors and of combinations thereof compared to the detection rate obtained from RGB sensor only, where “early time-points” are defined to be those time-points for which the symptoms are not yet visible by the naked eye (as judged by a trait expert), and as such are not expected to be distinguishable to a RGB sensor. Vice versa, “late time-points” are those time-points where an expert (and thus potentially an RGB sensor) seemed the symptoms to be visible.

TABLE 2
Detection of abiotic stress by single vs. plurality modals

| | Early Time Points | Late Time Points |
|---|---|---|
| Channel | (detection/accuracy) | (detection/accuracy) |
| RGB | Not significant | 70% |
| Thermal | Not significant | Not improved |
| Depth | Not significant | Not improved |
| RGB + Thermal | Detection | Not improved |
| RGB + multi- | Detection | Improved detection |
| spectral at | | |
| 670 nm | | |

As is evident from Table 2, using a plurality of sensors of different modalities (RGB and thermal senor, or RGB and multi-spectral sensor at 670 nm) enabled early detection of the biotic stress symptoms, which were not visible using RGB sensor only. The combination of RGB+670 nm readings not only enabled the detection, but improved its accuracy.

Example 2: Early detection of abiotic stress including registration steps

The above-described system related to banana plantlets and induction of abiotic stress by insufficient fertilization was used.

In this experiment, four stress regimens were applied:

Treatment A—No fertilizer (0%)—maximum stress

Treatment B—67% fertilizer

Treatment C—100% fertilizer

Treatment D—200% fertilizer

Further, in this experiment a combination of three imaging sensors was used: RGB camera, thermal camera (also referred to as InfraRed, IR), and depth camera. The camera used are as described in Example 1 hereinabove.

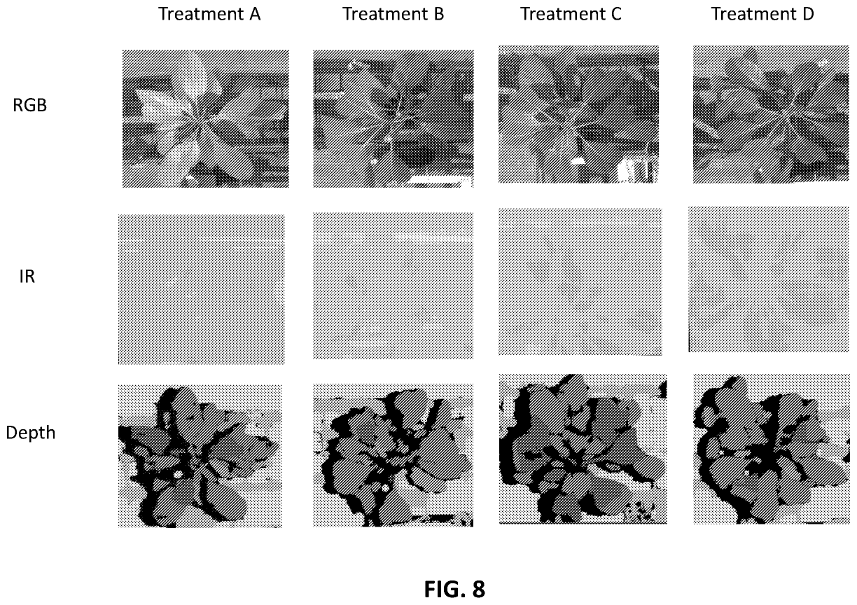

FIG. 8 shows images taken by RGB, IR and depth sensors, independently. This figure demonstrates that no significant difference is observed when the fertilizer was applied at different concentrations (67%, 100% or 200%). Accordingly, treatment “A” of 0% fertilizer was taken as inducing maximal stress, while treatments B-C were taken as not inducing stress on the examined plants.

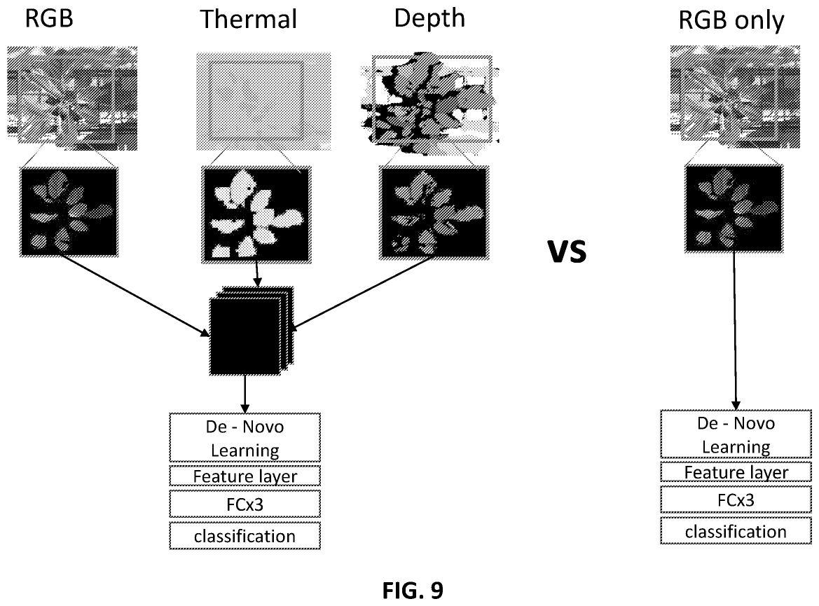

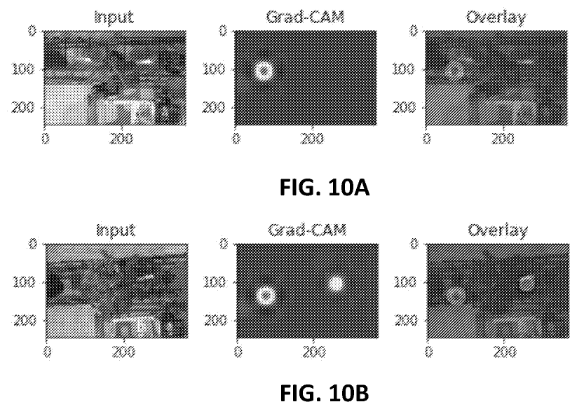

FIG. 9 shows an exemplary image of leaves taken with RGB sensor only and of images taken with multiple sensors including RGB, thermal and depth sensors and analyzed as described for FIG. 7 hereinabove. One problem in imaging of plants or other complicated objects, specifically in natural environment is that the image captures the object of interest as well as its surrounding. Using Gradient-weighted Class Activation Mapping (Grad-CAM) enables distinguishing the regions of input which form the basis for the phenotype predictions, in the current examples the banana leaves, from other objects of the image. FIGS. 10A and 10B demonstrate that using multiple sensors provides for better differentiation of the leaves (points or objects of interest), wherein in 42% of the cases the model identified the leaves (FIG. 10B), compared to only 26% in images taken by RGB only (FIG. 10A).

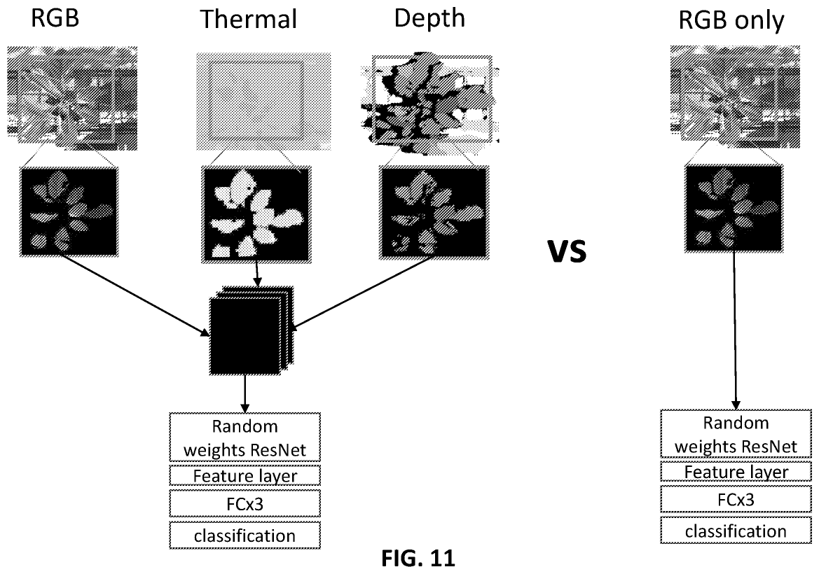

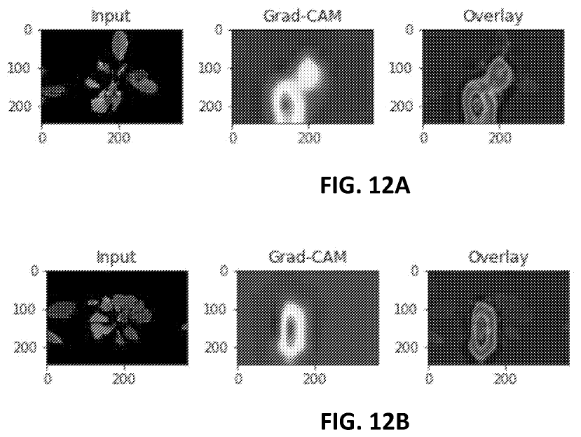

FIGS. 11 and 12 demonstrate registration of the images taken by multiple sensors vs. RGB sensor. The registration comprises masking in which the background of the RGB image is eliminated, such that artifacts of the surroundings are distinguishable from the leaves. Once registration is complete all images are aligned, and the background of the other images may be eliminated in accordance with the same mask. As is demonstrated in FIG. 12, such registration results in higher percentage of detection of the object of interest (93% detection using RGB, FIG. 12A vs. 96% using the multiple sensors FIG. 12B).

Table 3 demonstrates that using multiple imaging sensors provides significantly improved detection of stress resulting from lack of fertilizer, compared to images taken by RGB sensor only, at all the time points examined.

TABLE 3
Detection of abiotic stress by single vs. multi modals after registration

| | Early Time Points | Late Time Points |
|---|---|---|
| | (1-14 days) | (>14 days) |
| Channel | detection/accuracy) | (detection/accuracy) |
| RGB-masking | 58.9% ± 2.8 | 67.3% ± 7.9 |
| Multi-Modal: | 71.3% ± 4.2 | 79.8% ± 5.6 |
| RGB, Thermal, Depth | | |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A system for detecting or predicting a phenotype of a plant, comprising:
a plurality of imaging sensors of different modalities selected from the group consisting of: a Red-Green-Blue (RGB) sensor; a multispectral sensor; a hyperspectral sensor; a depth sensor; a time-of-flight camera; a LIDAR; and a thermal sensor, the plurality of sensors mounted on a bracket at predetermined geometrical relationships;
a computing platform comprising at least one computer-readable storage medium and at least one processor for:
receiving data captured by the plurality of sensors, the data comprising at least two images of at least one part of a plant, the at least two images captured at a distance of between 0.05 m and 10 m from the plant;
preprocessing the at least two images in accordance with the predetermined geometrical relationships, to obtain unified data;
extracting features from the unified data; and
providing the features to an engine to obtain a phenotype of the plant.

2. (canceled)

3. (canceled)

4. The system of claim 1, wherein the at least two images are captured at a distance of between 0.05 m and 5 m from the plant.

5. The system of claim 1, wherein the processor is further adapted to:
receive from at least one additional sensor additional data related to positioning and/or environmental conditions of the plant; and
process the at least two images using the additional data to eliminate effects generated by the environmental conditions and/or positioning to obtain at least two enhanced images before preprocessing.

6. The system of claim 5, wherein the preprocessing comprises preprocessing the at least two enhanced images.

7. The system of claim 5, wherein the at least one additional sensor is selected from the group consisting of a light sensor, a global positioning system (GPS), a digital compass, a radiation sensor, a temperature sensor, a humidity sensor, a motion sensor, an air pressure sensor, a soil sensor, an inertial sensor, and any combination thereof.

8. (canceled)

9. The system of claim 1, wherein said preprocessing comprises at least one of registration, segmentation, stitching, lighting correction, measurement correction, and resolution improvement.

10. The system of claim 9, wherein the preprocessing comprises registering the at least two enhanced images in accordance with the predetermined geometrical relationships.

11. (canceled)

12. (canceled)

13. The system of claim 1, wherein the computing platform is further configured to receive (i) information related to mutual orientation among the sensors; (ii) information related to mutual orientation between the sensors and at least one of an illumination source and the plant or a combination thereof

14. The system of claim 1, wherein the computing platform is further configured to receive information related to mutual orientation between the sensors and at least one of an illumination source and the plant.

15-23. (canceled)

24. The system of claim 5, further comprising a command and control unit for at least one of:
coordinating activation of the plurality of imaging sensors; and
operating the at least one processor in accordance with the plurality of imaging sensors and the at least one additional sensor.

25. The system of claim 24, wherein the command and control unit is further operative to perform at least one action selected from the group consisting of: setting a parameter of a sensor from the plurality of sensors; operating the at least one processor in accordance with a selected application; providing an indication to an activity status of a sensor from the plurality of sensors; providing an indication to a calibration status of a sensor from the plurality of sensors; and recommending to a user to calibrate a sensor from the plurality of sensors.

26. The system of claim 1, further comprising a communication unit for communicating data from said plurality of sensors to the computing environment.

27-31. (canceled)

32. The system of claim 1, wherein said system is implemented on a mobile phone comprising at least two imaging sensors of different modalities.

33. The system of claim 1, wherein the phenotype is selected from the group consisting of a biotic stress status, an abiotic stress status, a feature predicting harvest time, a feature predicting harvest yield, a feature predicting yield quality, and any combination thereof

34. The system of claim 1, wherein said system is further configured to generate as output data the phenotype, a quantitative phenotype, an agricultural recommendation based on said phenotype, or a combination of two or more thereof.

35. The system of claim 34, wherein the agricultural recommendation relates to at least one of yield prediction, monitoring male or female organs to estimate yield, monitoring fruit maturity, monitoring fruit size, monitoring number of fruit, monitoring fruit quality, nutrient management, and determining time of harvest.

36. The system of claim 34, wherein the computing platform is further configured to deliver the output data to a remote device of at least one user.

37. A system for training an engine for detecting or predicting a phenotype of a plant, comprising:
a plurality of imaging sensors of different modalities selected from the group consisting of: a Red-Green-Blue (RGB) sensor; a multispectral sensor; a hyperspectral sensor; a depth sensor; a time-of-flight camera; a LIDAR; and a thermal sensor, the plurality of sensors mounted on a bracket at predetermined geometrical relationships;
a computing platform comprising at least one computer-readable storage medium and at least one processor for:
receiving data captured by the plurality of sensors, the data comprising at least two images of at least one part of a plant, the at least two images captured at a distance of between 0.05 m and 10 m from the plant;
preprocessing the at least two images in accordance with the predetermined geometrical relationships, to obtain unified data;
obtaining annotations for the unified data, the annotations are associated with the phenotype of the plant; and
training an engine on the unified data and the annotations, to receive images of a further plant and determine or predict a phenotype of the further plant.

38. The system of claim 37, wherein training the engine is performed upon multiplicity of unified data obtained from images received at a plurality of time points or at a plurality of geographic locations.

39. A system for detecting or predicting a state of an object, comprising:
a plurality of imaging sensors of different modalities selected from the group consisting of: a Red-Green-Blue (RGB) sensor; a multispectral sensor; a hyperspectral sensor; a depth sensor; a time-of-flight camera; a LIDAR; and a thermal sensor, the plurality of sensors mounted on a bracket at predetermined geometrical relationships;
a computing platform comprising at least one computer-readable storage medium and at least one processor for:
receiving data captured by the plurality of sensors, the data comprising at least two images of at least one part of an object, the at least two images captured at a distance of between 0.05 m and 10 m from the object;
preprocessing the at least two images in accordance with the predetermined geometrical relationship, to obtain unified data;
extracting features from the unified data; and
providing the features to an engine to obtain a phenotype of the object.

40-49. (canceled)
